SEQUENCE LISTING (15 sequences)

SEQ ID NO: 1 - protein, 15 aa, Artificial sequence
  Synthetic peptide
AQREANKKIEKQLQK

SEQ ID NO: 2 - protein, 15 aa, Artificial sequence
  Synthetic peptide
NKKIEKQLQKDKQVY

SEQ ID NO: 3 - protein, 15 aa, Artificial sequence
  Synthetic peptide
KQLQKDKQVYRATHR

SEQ ID NO: 4 - protein, 15 aa, Artificial sequence
  Synthetic peptide
AGESGKSTIVKQMRI

SEQ ID NO: 5 - protein, 15 aa, Artificial sequence
  Synthetic peptide
KSTIVKQMRILHVNG

SEQ ID NO: 6 - protein, 15 aa, Artificial sequence
  Synthetic peptide
NPENQFRVDYILSVM

SEQ ID NO: 7 - protein, 14 aa, Artificial sequence
  Synthetic peptide
ILSVMNVDFDFPPE

SEQ ID NO: 8 - protein, 15 aa, Artificial sequence
  Synthetic peptide
NVPDFDFPPEFYEHA

SEQ ID NO: 9 - protein, 15 aa, Artificial sequence
  Synthetic peptide
ERRKWIQCFNDVTAI

SEQ ID NO: 10 - protein, 15 aa, Artificial sequence
  Synthetic peptide
IQCFNDVTAIIFVVA

SEQ ID NO: 11 - protein, 15 aa, Artificial sequence
  Synthetic peptide
LNLFKSIWNNRWLRT

SEQ ID NO: 12 - protein, 15 aa, Artificial sequence
  Synthetic peptide
EPGEDPRVTRAKYFI

SEQ ID NO: 13 - protein, 15 aa, Artificial sequence
  Synthetic peptide
PRVTRAKYFIRDEFL

SEQ ID NO: 14 - protein, 15 aa, Artificial sequence
  Synthetic peptide
AKYFIRDEFLRISTA

SEQ ID NO: 15 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

(19) United States
(12) Patent Application Publication
(10) Pub. No.: US 2008/0292627 A1
(43) Pub. Date: Nov. 27, 2008

(54) Compositions and Methods for Treating Mood and Anxiety Disorders

(75) Inventors: Mark M. Rasenick, Glenview, IL (US); Brian T. Layden, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE BOARD OF TRUSTEES OF THE UNIVERSITY OF ILLINOIS, Urbana, IL (US)

(21) Appl. No.: 11/913,404

(22) PCT Filed: May 5, 2006
(86) PCT No.: PCT/US06/17428

§ 371 Date: Jun. 23, 2008

Related U.S. Application Data
(60) Provisional application No. 60/678,050, filed on May 5, 2005.

(51) Int. Cl. A61K 39/395; C12N 5/06; G01N 33/53; A61P 43/00; A61K 38/00

(52) U.S. Cl. 424/135.1; 435/375; 514/2; 424/130.1; 514/14; 424/141.1; 435/7.2

(57) ABSTRACT

The invention relates method of disrupting the interface between Gsα and tubulin. The disruption of this interaction is a mechanism for treatment of mood and anxiety disorders and this interaction may be used to screen for and design therapeutics for mood and anxiety disorders.

Priority is claimed to U.S. Provisional Application No. 60/678,050 filed May 5, 2005, which is incorporated herein by reference in its entirety.

Certain of the studies described in the present application were conducted with the support of government funding in the form of a grant from the National Institutes of Mental Health, Grant No. MH039595 and MH00699. The United States government has certain rights in the invention.

### BACKGROUND OF INVENTION

1. Field of Invention

The invention relates to the disruption of the interface between Gs(alpha) and tubulin. The disruption of this interaction is a mechanism for the treatment of mood and anxiety disorders. The invention also provides for methods of screening for and the design of therapeutics for mood and anxiety disorders, such as depression.

2. Related Technology

Affective disorders are characterized by changes in mood as the primary clinical manifestation. Major depression is one of the most common mental illnesses and is often under diagnosed and frequently undertreated, or treated inappropriately. Major depression is characterized by feelings of intense sadness and despair, mental slowing and loss of concentration, pessimistic worry, agitation, and self-deprecation. Physical changes usually occur that include insomnia, anorexia and weight loss (or overeating) decreased energy and libido, and disruption of the normal circadian rhythms of activity, body temperature, and many endocrine functions. Many as 10-15% of individuals with this disorder display suicidal behavior during their lifetime.

Certain G protein a subunits, components of the G-protein Coupled Receptor (GPCR) signal transduction system, have been shown to form tight complexes with the cytoskeletal protein, such as tubulin. Microtubules, an essential component of the cytoskeleton, are composed of tubulin dimers where each dimer consists of an α and β monomer. One GTP binds to the nonexchangable site (N-site) on β-tubulin and another GTP binds at the exchangeable site (E-site) on β-tubulin. GTP hydrolysis occurs at the E-site when another dimer binds to the growing microtubule at the positive end (Carlier, Curr. Opin. Cell. Biol. 3: 12-17, 1991; Nogales, Annu. Rev. Biophys. Biomol. Struct. 30: 397-420, 2001). Over the past few years, significant progress has been made in the structural determination of the tubulin dimer. The domains on tubulin where drugs such as taxanes, colchine and vinblastine bind have been revealed. Much less information exists on where the microtubule associated proteins (MAPs) bind tubulin. However, many of the sites have been proposed to be on the C-terminus of tubulin. As the structure/function of tubulin dimers and microtubules is deciphered, more novel protein-protein interactions with tubulin will be determined (Nogales (2001), Annu. Rev. Biophys. Biomol. Struct. 30, 397-420).

G proteins are heterotrimeric structures composed of α, β, and γ subunits. Upon agonist binding to membrane receptors, the Gα subunit is activated by the exchange of GDP for GTP leading to the extracellular message being passed to the intracellular side (Lambright et al., Nature 369: 621-628, 1994). Activated Gα subunit interacts with effector proteins and allows Gβγ to interact with effectors as well. Recently, it has become apparent that Gα and Gβγ proteins interact with a vast array of other cellular proteins that can affect the G protein activation/deactivation cycle (Blumer et al., Receptors Channels 9, 195-204, 2003; Hepler, Mol. Pharmacol. 64: 547-549, 2003). Although distinct in structure and other properties from other G protein regulators, tubulin has long been known to interact with certain G proteins (Rasenick et al., Nature 294: 560-562, 1981; Wang et al., J. Biol. Chem. 265: 1239-1242, 1990). Of the Gα family of proteins, the inhibitory G protein α subunit of adenylyl cyclase (Giα1) and the stimulatory G protein α subunit of adenylyl cyclase (Gsα) bind with a high affinity to tubulin while other Gα subunits (e.g., the α subunit of the retinal G protein transducin; Gtα) show no measurable tubulin binding (Wang et al., J. Biol. Chem. 265: 1239-1242, 1990).

The protein-protein interaction between one of these G proteins, Gsα and tubulin has direct implications to the therapeutic mechanism by which antidepressants (and perhaps other drugs for mood and anxiety disorders) exert their effect. Studies suggest that chronic antidepressant treatment moves Gsα from a subcellular region enriched in tubulin to one where Gsα becomes less associated with tubulin. In these regions, Gsα also engages in a more facile activation of adenylyl cyclase. Elucidation of the binding sites between Gα subunits and tubulin dimers will provide insight into this complex and interaction and the biochemical mechanism of antidepressant therapy.

Antidepressant therapies are present in many diverse forms, including tricyclic compounds, monoamine oxidase inhibitors, selective serotonin reuptake inhibitors (SSRIs), atypical antidepressants, and electroconvulsive treatment. There remains a need for the identification and development of new antidepressant therapies, as well as methods for screening for novel antidepressant agents.

### SUMMARY OF INVENTION

It is known that tubulin interacts with many different proteins (Nogales, Annu. Rev. Biophys. Biomol. Struct. 30: 397-420,2001). Structural information on these protein interactions has been limited except in a few cases (Nogales, Annu. Rev. Biophys. Biomol. Struct. 30: 397-420, 2001; Gigant et al., Cell 102: 809-816, 2000). Many of these proteins, in particular Tau proteins, appear to interact with tubulin through the acidic C-terminal of tubulin (Nogales, Annu. Rev. Biophys. Biomol. Struct. 30: 397-420, 2001, 2001). This model described herein suggests that a binding site for Gα at the plus end of a microtubule which is quite different from that of MAPs. As seen in FIGS. 3-5, the nucleotide-binding pocket and the surrounding residues of β-tubulin comprise a majority of the Gsα interacting surface. Further, the Gsα molecule completely encases the nucleotide-binding pocket of β-tubulin. Gsα appears to be the first protein identified to associate with β-tubulin at the GTP binding site.

In one embodiment, the invention provides for methods of disrupting the complex of Gsα and tubulin in a cell comprising inhibiting the interaction of Gsα and tubulin within said cell by contacting tubulin with a molecule that inhibits the interaction of Gsα and tubulin, wherein the molecule binds to tubulin within at least one region selected from the group consisting of the nucleotide binding site, H1 region or H2 region of tubulin.

In another embodiment, the invention provides for methods of disrupting the complex of Gsα and tubulin in a cell comprising inhibiting the interaction of Gsα and tubulin within said cell by contacting Gsα with a molecule that inhibits the interaction of Gα and tubulin, wherein the molecule binds to Gsα within at least one region selected from the group consisting of the α3-β5 region, α4-β6 region, α2-β4 region or the amino terminus of Gsα.

The invention also provides for compositions comprising a molecule that inhibits the interaction of Gsα and tubulin. These molecules include those that bind tubulin within at least one region selected from the group consisting of the nucleotide binding site, H1 region or H2 region of tubulin. These molecules also include those that bind Gsα within at least one region selected from the group consisting of α3-β5 region, α4-β6 region, α2-β4 region or the amino terminus of Gsα.

The molecules that inhibit the interaction of Gsα and tubulin are peptides, small molecules, antibodies, including single-chained antibodies, and monoclonal antibodies or peptidomimetics. Peptides of the invention include peptide comprising an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14.

In a further embodiment, the invention provides for methods of treating a mood or anxiety disorder comprising administering a composition comprising a molecule that inhibits the interaction of Gsα and tubulin.

In another embodiment, the invention provides for methods of identifying modulators of the interaction of Gsα and tubulin comprising contacting a cell expressing Gsα and tubulin with a candidate compound, and monitoring said cell for modulation of Gsα binding to tubulin, wherein a candidate compound that reduces the binding of Gsα to tubulin is an inhibitor of the interaction of Gα and tubulin, and a candidate compound that increase the binding of Gsα to tubulin is a agonist of the interaction of Gsα and tubulin.

### DESCRIPTION OF DRAWING

FIGS. 1A and 1B provides comparison of tubulin binding regions for Gsα, Giα and Gtα). FIG. 1A is an alignment of tubulin binding region on the Sequence of Gsα. Amino acid sequences for the Gsα peptide spots along with the corresponding sequences of Giα and Gtα that exhibited binding to tubulin are indicated for tubulin in the GDP state (shown in italics), GTP state (shown in bold) as well as the domains that showed binding to tubulin in both nucleotide states (underlined).The specific domains, as α-helices (α) or β-sheets (β) as well as their corresponding number or letter are indicated as described before (Sunahara et al., Science 278: 1943-1947, 1997). The secondary structure of Gsα as represented by rectangles (for α-helices) or arrowheads (for β-sheets) is indicated above the amino acid sequences. FIG. 1B depicts binding of tubulin to Gsα-peptides as compared to Gtα-Peptides. Peptides from Gsα showing tubulin binding were compared to the corresponding Gtα peptide. The membrane was incubated with 150 nM tubulin-GDP or tubulin-GTP. There are some sequence alignment differences between this and FIG. 1A, because Gsα-Gtα sequences were aligned here, whereas in FIG. 1A, all three sequences (Gsα, Giα1, and Gtα) were aligned together. Furthermore, for spot 63, 65, and 67, residues in bold are those of Gsα, as this sequence is missing for Gtα.

FIG. 2 depicts the tubulin binding regions on the tertiary structure of Gsα. Stereo structure of Gsα-GTPγS in a line ribbon form (Sunahara et al., Science 278: 1943-1947, 1997) with the regions showing binding to β-tubulin highlighted in the colors described in FIG. 1 (blue for tubulin-GDP binding site, red for tubulin-GTP binding site, green for tubulin binding in both nucleotide states). The side chain of TRP281 and GTPγS are shown in the ball and stick configuration with the color scheme by atom type. The structure of Gsα-GTPγS (Sunahara et al., Science 278: 1943-1947, 1997) is missing the following residues determined to be important from the membrane binding: ALA18-LYS34 and ASN66-PHE68.

FIGS. 3A and 3B depicts the relative orientation of β-Tubulin and Gsα for the Top 30 Complexes, calculated by ZDOCK and ClusPro. FIG. 3A depicts stereo representation of the orientation of β-tubulin relative to a fixed Gsα-GTPγS, in which the backbone is rendered as a tube, for the top 30 complexes. Gsα-GTPγS is shown as described in FIG. 2 with the regions of Gsα-GTPγS showing binding to tubulin highlighted. A circle at its geometric center represents β-tubulin for each of the 30 complexes. A color bar on the figure corresponds to the ranking of the complexes from 1 to 30, with red indicating highest ranked complex, blue indicating lowest ranked complex. Complex four is indicated by number “4”. FIG. 3B depicts stereo representation of the orientation of Gsα-GTPγS relative to a fixed β-tubulin, in which the backbone is rendered as a tube, for the top 30 complexes. A circle at its geometric center represents the Gsα molecules for each of the 30 complexes. A color bar on the figure corresponds to the ranking of the complexes from 1 to 30, with red indicating highest ranked complex, blue indicating lowest ranked complex. The highlighted residues in green on β-tubulin represent the predicted interface with Gsα. Complex four is indicated by number “4”.

FIG. 4 provides representations of complex 1-5 for Gsα-β-tubulin interaction. Both proteins are presented in the α carbon form, where Gsα is in white and fixed in orientation to the position of the β-tubulin molecules for each of the top 5 complexes. β-tubulin molecules for the different complexes are in: red (complex 1), yellow (complex 2), green (complex 3), blue (complex 4) and purple (complex 5).

FIG. 5 depicts a ribbon representation of complex 2. Gsα and tubulin are displayed in the a carbon backbone form in green-blue and β-tubulin is displayed in the α carbon backbone in red. Important interacting domains on Gsα and β-tubulin are labeled. The nucleotides are shown in the ball and stick configuration with the color scheme by atom type.

### DETAILED DESCRIPTION

Despite several decades of studies, the mechanism of antidepressant action has not been clearly established. One of the most widely known biochemical effects of antidepressant treatment is an alteration in the density and/or sensitivity of several neurotransmitter receptor systems (Sulser, Adv. Biochem. Psychopharmacol., 39:249-261; 1984). However, these effects do not fully explain the clinical efficacy of all antidepressants, mainly because of the dissociation between the time course of the change in the receptor numbers and their clinical time course (Rasenick et al., J. Clin. Psychiatry, 57:49-55; 1996).

Many studies searching for a common mechanism of antidepressant action have focused on postreceptor neuronal cell signaling processes as potential targets of such action (Menkes et al. Science, 219:65-76, 1983; Ozawa et al., Mol. Pharmacol., 36:803-808, 1989; Duman et al., Arch. Gen. Psychiatry, 54:597-606, 1997; Takahashi et al., J. Neurosci., 19:610-616, 1999). Much of this previous work has focused on the downstream effects of antidepressant action, particularly those involving cAMP (Perez et al., Eur. J. Pharmacol., 172:305-316, 1989; Perez et al., Neuropsychopharmacology, 4:57-64, 1991; Duman et al., Arch. Gen. Psychiatry, 54:597-606, 1997; Takahashi et al., J. Neurosci., 19:610-616, 1999).

Much of the current thinking about G protein-coupled receptors is based on the idea of freely mobile receptors, G proteins, and effectors in which the specificity of their interaction is derived from the three-dimensional structure of the sites of protein-protein interactions. However, recent evidence indicates that an organized interaction of receptors, G proteins, and effectors with significant limitations on lateral mobility (Kuo et al., Science, 260:232-234; 1993). Furthermore, these membrane proteins are associated with tubulin or other cytoskeletal proteins (Carlson et al., Mol. Pharmacol., 30:463-468, 1986; Rasenick et al., Adv. Second Messenger Phosphoprotein Res., 22:381-386, 1990; Wang et al., Biochemistry, 30:10957-10965, 1991), which restrict distribution and mobility of G proteins to a surprising degree (Neubig, FASEB, 8:939-946; 1994). The presence of a well organized network of cytoskeletal elements and the components of neurotransmitter and hormonal G protein-mediated signal transduction systems may play an important role in achieving this function.

### Tubulin-Gsα Interaction

Tubulin-Gα interaction has been shown to induce changes in the GTP/GDP binding and kinetics in both Gα and tubulin. G proteins binding to tubulin activate the GTPase activity of tubulin, destabilizing the microtubules (Roychowdhury et al., J. Biol. Chem. 274: 13485-13490, 1999). Conversely, G proteins can be activated in a receptor independent mechanism in which a direct transfer of GTP (transactivation) from the E-site on tubulin to the Gα subunit occurs (Rasenick et al., Methods Enzymol. 390: 389-403, 2004). In the case of Gsα, this receptor-independent activation of Gsα subunits increases the coupling of Gsα to adenylyl cyclase (Yan et al., J Neurochem. 76: 182-190, 2001). Studies using chimeric proteins of Giα1 and Gtα to disrupt tubulin-Gα interaction, demonstrated that residues 237-270 of Giα1 (which corresponds to residues 253-293 in Gsα) are crucial for the transactivation of Gα by tubulin and also play a role in modulating microtubule organization and elongation of cellular processes. Elucidation of the binding sites between Gα subunits and tubulin dimers provides insight into this complex and novel interaction.

Crystallographic studies have provided an excellent method to determine protein-protein structures. However, tubulin has been difficult to study by crystallographic approaches (Nogales et al., Nature 391: 199-203, 1998; Gigant et al., Cell 102: 809-816, 2000; Mandelkow et al., Nature 287: 595-599, 1980). Molecular docking programs along with verification through biochemical assays, as described herein, are another approach for determining protein-protein structures.

The model of Gsα and tubulin interaction described herein was developed using a combination of biochemical and molecular docking techniques. Overlapping peptides were covalently attached via the primary amino acid Gsα sequence to a membrane and tubulin binding to specific spots on the membrane was determined (Frank, J. Immunol. Methods 267: 13-26, 2002). This study identified potential high affinity sites important for tubulin-Gsα interaction. (see Example 2). In addition, protein-protein docking algorithms were used to generate and refine a model for the interacting facets of these molecules (Chen, et al., Proteins 52: 68-73, 2003; Comeau et al., Bioinformatics 20: 45-50, 2004). (see Example 3)

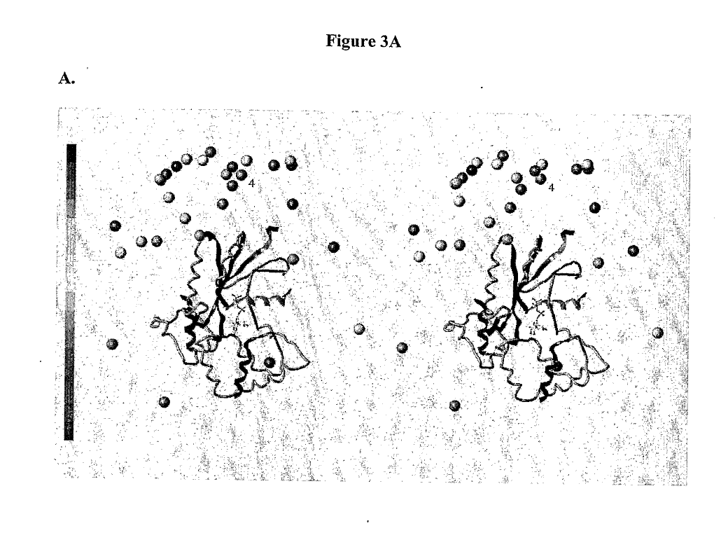

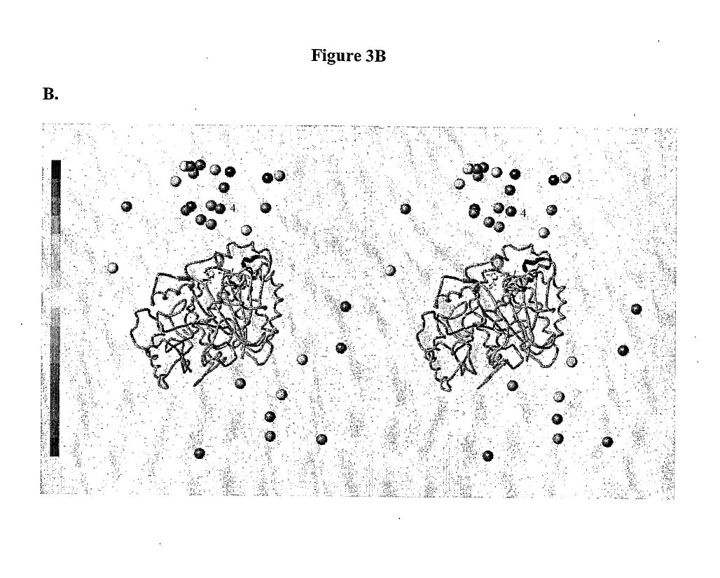

The relative position of β-tubulin to a fixed Gsα protein is shown in FIG. 3a. This binding position of tubulin is within the GTPase domain of Gsα. The relative position of Gsα to a fixed β-tubulin protien is shown in FIG. 3b. The binding position of Gα to β-tubulin is within the exchangable nucleotide-binding site of β-tubulin and the H1 (α-helix 1) and H2 (α-helix 2) regions of β-tubulin. In addition, common contact regions between Gsα and β-tubulin are within the α3-β5 (switch III), and the α4-β6 loop of Gsα, the α2-β4 (switch II) region of Gsα and the amino terminal of Gsα.

The model of Gsα and tubulin interaction described herein is the first report of a structural models of the Gsα-tubulin complex and suggests that tubulin interacts with Gsα predominantly in the GTPase domain, more precisely with regions essential to adenylyl cyclase activation (α2-β4 and α3-β5) (Tesmer et al., Science 278: 1907-1916, 1997). This model also suggests that Gsα binds to tubulin such that it surrounds the nucleotide-binding site of β-tubulin, in a region of tubulin normally involved in docking other tubulin molecules during microtubule polymerization. These structures reveal how tubulin might transactivate Gsα and how Gsα can activate tubulin GTPase.

The structural interaction between tubulin and Gsα described herein has implications for the function of each protein. The domains on Gsα (α2-β4 and α3-β5) that are essential to the binding and activation of adenylyl cyclase (Tesmer et al., Science 278: 1907-1916, 1997) are also important for the interaction with tubulin. These observations indicate how Gsα-tubulin interaction may alter the interaction of Gsα with adenylyl cyclase (Wang et al., J. Biol. Chem. 265: 1239-1242, 1990; Yan et al., J Neurochem. 76: 182-190, 2001; Roychowdhury et al., J. Biol. Chem. 274: 13485-13490, 1999; Rasenick, et al., Methods Enzymol. 390: 389-403, 2004).. Further, these observations provide a structural basis to the finding that Gsα interacts with tubulin at the exchangeable site on β-tubulin activating the GTPase of tubulin and increasing dynamics of microtubules (Roychowdhury et al., J. Biol. Chem. 274: 13485-13490, 1999). Gsα appears to surround the exposed GTP on the β-tubulin, which includes the region of the GTP cap of microtubules.

Gsα facilitates GTP hydrolysis on tubulin, which leads to microtubule depolymerization by increased GTPase activation on tubulin (Roychowdhury et al., J. Biol. Chem. 274: 13485-13490, 1999). This is consistent with the structural models described herein. The experimental and theoretical analyses described herein provides the first proposed structural model for the Gsα-tubulin complex.

### Disruption of Tubulin-Gsα Interaction

Disruption of the tubulin-Gsα complex is contemplated to be useful as a therapeutic mechanism for treating and preventing mood and anxiety disorders. It is known that antidepressant therapy induces a shift in the subcellular localization of Gsα from a region enriched with tubulin to a region less associated with tubulin. Thus, molecules that induce this shift in Gsα subcellular localization or those that disrupt the interaction of tubulin with Gsα are contemplated as therapeutics for preventing or ameliorating mood and anxiety disorders, such as depression.

The invention contemplates as therapeutics for mood and anxiety disorders, any molecule that disrupts or inhibits the formation of the Gsα-tubulin complex. These therapeutic molecules include peptides, small molecules, antibodies and fusion or chimeric proteins. Any of the therapeutic compositions described below can be used alone or in combination with each other. Further, the present invention also contemplates the use of the following compositions in combination with standard treatments presently being used for the treatment of mood and anxiety disorders, such as known antidepressants, such as tricyclic compounds (e.g. amitriptyline, clomipramine, amitriptyline, amitriptyline, maprotiline, desipramine, nortryptyline, desipramine, doxepin, trimipramine, imipramine, protriptyline), monoamine oxidase inhibitors (e.g. phenelzine, tranylcypromine), selective serotonin reuptake inhibitors (SSRIs) (e.g. citalopram, escitalopram oxalate, fluvoxamine, paroxetine, fluoxetine, sertraline), atypical antidepressants and electroconvulsive treatment.

### A. Peptides

The present invention provides peptides that may be used to disrupt the Gsα-tubulin complex. The invention particularly provides peptides that specifically bind within the Gsα binding site on tubulin or the tubulin binding site on Gsα. Exemplary peptides that bind tubulin within the Gsα binding site are those set out in Table 1.

TABLE 1

| | | |
|---|---|---|
| peptide 1 | AQREANKKIEKQLQK | SEQ ID NO: 1 |
| peptide 2 | NKKIEKQLQKDKQVY | SEQ ID NO: 2 |
| peptide 3 | KQLQKDKQVYRATHR | SEQ ID NO: 3 |
| peptide 4 | AGESGKSTIVKQMRI | SEQ ID NO: 4 |
| peptide 5 | KSTIVKQMRILHVNG | SEQ ID NO: 5 |
| peptide 6 | NPENQFRVDYILSVM | SEQ ID NO: 6 |
| peptide 7 | ILSVMNVPDFDFPPE | SEQ ID NO: 7 |
| peptide 8 | NVPDFDFPPEFYEHA | SEQ ID NO: 8 |
| peptide 9 | ERRKWIQCFNDVTAI | SEQ ID NO: 9 |
| peptide 10 | IQCFNDVTAIIFVVA | SEQ ID NO: 10 |
| peptide 11 | LNLFKSIWNNRWLRT | SEQ ID NO: 11 |
| peptide 12 | EPGEDPRVTRAKYFI | SEQ ID NO: 12 |
| peptide 13 | PRVTRAKYFIRDEFL | SEQ ID NO: 13 |
| peptide 14 | AKYFIRDEFLRISTA | SEQ ID NO: 14 |

The peptides, set out in Table 1, were designed using the immobilized-peptide array technique described in Example 2. The invention contemplates other peptides that bind Gsα within the GTPase domain, including the switch regions of Gsα (switch I, switch II and switch III). For example, the invention contemplates peptides that bind to α3-β5 region of Gsα or the α2-β4 (switch II region) of Gsα. Other exemplary peptides include peptides that bind to Ala18-Lys34 of the Gsα amino acid sequence or peptides that bind to Asn66-Phe68 of the Gsα amino acid sequence or peptides that bind to amino acids 253-293 of the Gsα amino acid sequence are contemplated. The invention also contemplates peptides that bind within the relative position of Gsα as shown in FIG. 3a.

Peptides that bind to Gsα and thereby inhibit its binding to tubulin are also contemplated as therapeutic molecules of the invention. The invention also contemplates peptides that bind to the amino terminus of Gsα. For example, the invention contemplates peptides that bind near the nucleotide binding site on tubulin, H1 region of tubulin and the H2 region of tubulin. The invention also contemplates peptides that bind within the relative position of β-tubulin as shown in FIG. 3b.

In discussing the sequences of the peptides of the invention, the present application employs the conventional abbreviations for the amino acids as follows: Alanine, Ala, A; Arginine, Arg, R; Asparagine, Asn, N; Aspartic acid, Asp, D; Cysteine, Cys, C; Glutamine, Gln, Q; Glutamic Acid, Glu, E; Glycine, Gly, G; Histidine, His, H; Isoleucine, Ile, I; Leucine, Leu, L; Lysine, Lys, K; Methionine, Met, M; Phenylalanine, Phe, F; Proline, Pro, P; Serine, Ser, S; Threonine, Thr, T; Tryptophan, Trp, W; Tyrosine, Tyr, Y; Valine, Val, V; Aspartic acid or Asparagine, Asx, B; Glutamic acid or Glutamine, Glx, Z; Norleucine, Nle; Acetyl-glycine (Ac)G; Any amino acid, Xaa, X. Additional modified amino acids known to those of skill in the art also may be used.

The peptides of the invention may be tested for their ability to bind to tubulin of Gsα using the SPOT membrane assays as described in detail in Example 1. Another method to determine the interactions between tubulin and G proteins as well as the binding of peptides to either tubulin or G proteins and the effect of those peptides on the interaction between tubulin and G proteins is surface plasmon resonance.

The peptide of the present invention may be any length of amino acids so long as the amino acids are of a sufficient length to interfere with the interaction of Gsα and tubulin. Preferably, the novel peptide inhibitors of the Gsα-tubulin interaction are at least about five amino acids in length, in certain embodiments the novel peptides of the present invention may comprise a contiguous amino acid sequence of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more amino acids.

In considering the particular amino acid to be positioned at any of the positions of the peptide, it may be useful to consider the hydropathic index of amino acids at each of the positions in a peptide known to be an effective inhibitor of the Gsα-tubulin interaction, and substitute a given amino acid with one of a similar hydropathic index. It is accepted that the relative hydropathic character of the amino acid contributes to the secondary structure of a resultant protein or peptide, which in turn defines the interaction of that protein with other molecules, for example, enzymes, substrates, receptors, DNA, antibodies, antigens, and the like. Each amino acid has been assigned a hydropathic index on the basis of their hydrophobicity and charge characteristics (Kyte & Doolittle, J. Mol. Biol., 157(1):105-132, 1982, incorporated herein by reference). Generally, amino acids may be substituted by other amino acids that have a similar hydropathic index or score and still result in a protein with similar biological activity i.e., still obtain a biological functionally equivalent protein or peptide. This is known as a conservative amino acid substitution. In the context of the peptides of the present invention, a biologically functionally equivalent protein or peptide will be one which still retains its ability to be an antagonist of the Gsα binding to tubulin or an antagonist of tubulin binding Gsα.

In addition, the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As such, an amino acid can be substituted for another having a similar hydrophilicity value and still obtain a biologically equivalent and immunologically equivalent protein.

In preferred aspects of the invention, peptides are synthesized according to methods known to those of skill in the art (Carter, et al., Biotechnology, 10(5): p. 509-13, 1992; Chen, et al., in Peptides: Wave of the Future—Proceedings of the 17th American Peptide Symposium, ed. M. Lebl and R. Houghten, Editors., Mayflower Scientific Ltd.: Kingswinford, UK. pp 206-207 and pp 318-319, 2001. In addition, short peptides sequences may be prepared by chemical synthesis using standard means. Particularly convenient are solid phase techniques (see, e.g., Erikson et al., The Proteins (1976) v. 2, Academic Press, New York, p. 255). Automated solid phase synthesizers are commercially available. In addition, modifications in the sequence are easily made by substitution, addition or omission of appropriate residues. The peptides of the present invention can also be produced by recombinant techniques. The coding sequence for peptides of this length can easily be synthesized by chemical techniques, e.g., the phosphotriester method described in Matteucci et al., J Am. Chem. Soc., 103: 3185, 1981.

In addition to the novel peptide inhibitors described above, the present invention further contemplates the generation terminal additions, also called fusion proteins or fusion polypeptides, of the peptides described above or identified according to the present invention. This fusion polypeptide generally has all or a substantial portion of the native molecule (i.e., the peptide inhibitors discussed above), linked at the N- and/or C-terminus, to all or a portion of a second or third polypeptide. It is contemplated that the fusion polypeptide may be produced by recombinant protein production or by automated peptide synthesis.

General principles for designing and making fusion proteins are well known to those of skill in the art. For example, fusions typically employ leader sequences from other species to permit the recombinant expression of a protein or peptide in a heterologous host. Another useful fusion includes the addition of an immunologically active domain, such as an antibody epitope, to facilitate purification of the fusion polypeptide. Inclusion of a cleavage site at or near the fusion junction will facilitate removal of the extraneous polypeptide after purification. The recombinant production of these fusions is described in further detail elsewhere in the specification. Other useful fusions include linking of functional domains, such as active sites from enzymes, glycosylation domains, cellular targeting signals or transmembrane regions.

There are various commercially available fusion protein expression systems that may be used to provide a tagged sequence in this context of the present invention. Particularly useful systems include but are not limited to the glutathione S-transferase (GST) system (Pharmacia, Piscataway, N.J.), the maltose binding protein system (NEB, Beverley, Mass.), the FLAG system (IBI, New Haven, Conn.), and the 6xHis system (Qiagen, Chatsworth, Calif.). These systems are capable of producing recombinant polypeptides bearing only a small number of additional amino acids, which are unlikely to affect the biologically relevant activity of the recombinant fusion protein. For example, both the FLAG system and the 6xHis system add only short sequences, both of which are known to be poorly antigenic and which do not adversely affect folding of the polypeptide to its native conformation. Another N-terminal fusion that is contemplated to be useful is the fusion of a Met-Lys dipeptide at the N-terminal region of the protein or peptides.

In addition to creating fusion polypeptides, it is contemplated that the fusion proteins or the peptide inhibitors may be further modified to incorporate, for example, a label or other detectable moiety.

Preferred peptide will comprise internally quenched labels that result in increased detectability after cleavage of the peptide inhibitors. The peptide inhibitors may be modified to have attached a paired fluorophore and quencher including but not limited to 7-amino-4-methyl coumarin and dinitrophenol, respectively. Other paired fluorophores and quenchers include bodipytetramethylrhodamine and QSY-5 (Molecular Probes, hIc.). In a variant of this assay, biotin or another suitable tag may be placed on one end of the peptide to anchor the peptide to a substrate assay plate and a fluorophore may be placed at the other end of the peptide. Useful fluorophores include those listed above as well as Europium labels such as W8044 (EG&g Wallac, Inc.).

Further, the peptides may be labeled using labels well known to those of skill in the art, e.g., biotin labels are particularly contemplated. The use of such labels is well known to those of skill in the art and is described in, e.g., U.S. Pat. No. 3,817,837; U.S. Pat. No. 3,850,752; U.S. Pat. No. 3,996,345 and U.S. Pat. No. 4,277,437. Other labels that will be useful include but are not limited to radioactive labels, fluorescent labels and chemiluminescent labels. U.S. Patents concerning use of such labels include for example U.S. Pat. No. 3,817,837; U.S. Pat. No. 3,850,752; U.S. Pat. No. 3,939,350 and U.S. Pat. No. 3,996,345. Any of the peptides of the present invention may comprise one two or more of any of these labels.

### B. Small Molecules and Antibodies

Disruption of the Gsα-tubulin complex can also be accomplished through the use of an organochemical composition (i.e., a small molecule inhibitor) that interferes with the Gsα binding to tubulin or tubulin binding to Gsα, by use of an antibody that blocks an Gsα active site or the Gsα binding site on tubulin or tubulin binding Gsα, or by use of a molecule that mimics the tubulin target (tubulin or Gsα).

With respect to small molecule inhibitors such compounds may be identified through standard screening assays. In particular, small molecules of the invention may be designed or developed based on the peptides set out in Table 1. Various candidate substances can be contacted with Gsα followed by further determination of the ability of treated Gsα to bind to tubulin. An agent that inhibits such binding will be a useful for blocking the Gsα-tubulin interaction. Alternatively, small molecules that bind to tubulin and block binding to Gsα are also contemplated.

The present invention provides for antibodies and antibody fragments that bind to tubulin or Gsα and antagonize the Gsα-tubulin interaction. The invention also provides for antibodies that bind to the tubulin or Gsα and induce a conformational change that prevents Gsα-tubulin interaction. The antibodies may be polyclonal including monospecific polyclonal, monoclonal (mAbs), recombinant, chimeric, humanized such as CDR-grafted, human, single chain, and/or bispecific, as well as fragments, variants or derivatives thereof. Antibody fragments include those portions of the antibody which bind to an epitope on tubulin or Gsα. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions. The methods by which antibodies are generated are well known to those of skill in the art.

A particularly useful antibody for disrupting the Gsα-tubulin interaction is a single chain antibody. Methods for the production of single-chain antibodies are well known to those of skill in the art. The skilled artisan is referred to U.S. Pat. No. 5,359,046, (incorporated herein by reference) for such methods. A single chain antibody, preferred for the present invention, is created by fusing together the variable domains of the heavy and light chains using a short peptide linker, thereby reconstituting an antigen binding site on a single molecule.

Single-chain antibody variable fragments (Fvs) in which the C-terminus of one variable domain is tethered to the N-terminus of the other via a 15 to 25 amino acid peptide or linker, have been developed without significantly disrupting antigen binding or specificity of the binding. These Fvs lack the constant regions (Fc) present in the heavy and light chains of the native antibody.

Polyclonal antibodies of the invention generally are produced in animals (e.g., rabbits or mice) by means of multiple subcutaneous or intraperitoneal injections of the target protein of interest and an adjuvant. It may be useful to conjugate a target peptide to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for antibody titer.

Monoclonal antibodies are produced using any method which provides for the production of antibody molecules by continuous cell lines in culture. Examples of suitable methods for preparing monoclonal antibodies include the hybridoma methods of Kohler et al., Nature, 256:495-497 (1975) and the human B-cell hybridoma method, Kozbor, J. Immunol., 133:3001 (1984); Brodeur et al., Monoclonal Antibody Production Techniques and Applications, pp. 51-63 (Marcel Dekker, Inc., New York, 1987). Also provided by the invention are hybridoma cell lines which produce monoclonal antibodies reactive with target peptides of the invention.

With respect to inhibitors that mimic tubulin targets, the use of mimetics provides one example of custom designed molecules. Such molecules may be small molecule inhibitors that specifically inhibit Gsα binding to tubulin or tubulin binding to Gsα. Such molecules may be sterically similar to the actual target compounds, at least in key portions of the target's structure and or organochemical in structure. Alternatively these inhibitors may be peptidyl compounds, these are called peptidomimetics. Peptide mimetics are peptide-containing molecules which mimic elements of protein secondary structure. The underlying rationale behind the use of peptide mimetics is that the peptide backbone of proteins exists chiefly to orient amino acid side chains in such a way as to facilitate molecular interactions, such as those of ligand and receptor. An exemplary peptide mimetic of the present invention would, when administered to a subject, bind Gsα in a manner analogous to the tubulin domain binding to wild-type Gsα.

Successful applications of the peptide mimetic concept have thus far focused on mimetics of β-turns within proteins, which are known to be highly antigenic. Likely β-turn structures within an antigen of the invention can be predicted by computer-based algorithms as discussed above. Once the component amino acids of the turn are determined, mimetics can be constructed to achieve a similar spatial orientation of the essential elements of the amino acid side chains.

In a preferred embodiment, the present invention will provide an agent that binds competitively to tubulin at the Gsα binding site. In a more preferred embodiment, the agent will have an even greater affinity for tubulin than Gsα does. Affinity for tubulin can be determined in vitro by performing kinetic studies on binding rates.

In a preferred embodiment, the present invention will provide an agent that binds competitively to Gsα at the tubulin binding site. In a more preferred embodiment, the agent will have an even greater affinity for Gsα than tubulin does. Affinity for Gsα can be determined in vitro by performing kinetic studies on binding rates.

Other compounds may be developed based on computer modeling and predicted higher order structure, of the heterotrimeric complex of Gsα molecule and tubulin, such as the sites identified in FIGS. 3 and 3b. This approach has proved successful in developing inhibitors for a number of receptor/ligand interactions. The detailed structural information of the complex can guide one to build specific small chemical entities that interact with this site. One such a structure-based approach is to use nuclear magnetic resonance (NMR) spectroscopy and a linked-fragment approach to identify chemical leads for development of specific ligands for therapeutic targets (Hajduk et al., Science, 278, 498-499, 1997; Moore, Curr Opin in Biotech., 10, 54-58, 1999; Pellecchia et al., Nature Reviews Drug Discovery, 1, 211-219, 2002). The novel ligands are chemical entities constructed from building blocks identified from NMR-based screening and optimized for binding to a target protein.

The NMR-based chemical compound screening has significant advantages that make it preferable even over the newest methods of high-throughout screening of natural products or combinatorial chemical libraries (Fejzo et al., Chem. Biol. 6, 755-769, 1999; Hajduk et al., J Am Chem Soc 119, 5818-5827, 1997; Hajduk et al., Bioorganic & Medicinal Chemistry Letters 9, 2403-2406, 1999; Pellecchia et al., J Biomol NMR 22, 165-173, 2002). These unique advantages include (i) structure-based and selective screening for specific sites on a target protein; (ii) rapid and reliable screening of weak binding ligands; (iii) a large virtual library of small chemical compounds; and (iv) independent optimization of individual chemical fragments. The resulting linked chemical compounds with high affinity and selectivity are then subject to detailed structure-based analysis of their interactions with the target protein using a combination of NMR and computational modeling techniques. Refinement, chemical diversification through various chemical linkages and selectivity enhancement are achieved at this stage.

For a detailed description of methods for identification of small molecule inhibitors those of skill in the art are referred to WO 01/51521, which describes the three-dimensional structure of a complex between phosphotyrosine binding domain of Suc1-associated neurotrophic factor target protein and the SNT binding site of fibroblast growth factor receptor. Rational drug design predicated on the three-dimensional structure of this interaction is described in detail. It is contemplated that the techniques therein may be used for rational drug design to identify agents that can inhibit the deleterious effects of Gsα binding to tubulin. For example such a method would involve identifying a compound that destabilizes the Gsα-tubulin complex and would involve obtaining a set of atomic coordinates that define the three dimensional structure of a Gsα-tubulin complex. These coordinates are determined using a complex which comprises an tubulin protein interacting with a Gsα protein (Gsα-tubulin complex). The next step involves performing rational drug design with the atomic coordinates to select a drug that interferes with the Gsα-tubulin complex at a given site, such as the binding site. This rational drug design is preferably performed in conjunction with computer modeling. Upon selection of the candidate drug, the candidate is contacted with a Gsα-tubulin complex comprising a full length or fragment of tubulin protein and a full length or fragment of a Gsα protein. The stability of the Gsα-tubulin complex is monitored in the presence and absence of the candidate substance to identify a potential therapeutic agent which destabilizes the complex. Similar methods may be performed to identify a compound which inhibits the formation of the complex. Such methods are described in detail in WO 01/51521.

Methods of Screening for Compounds that Disrupt the Gsα-Tubulin Complex

The present invention also contemplates screening for compounds that disrupt the interaction of Gsα-tubulin complex. These compounds are contemplated to be potential antidepressant agents or therapeutics for mood or anxiety disorders.

This realization affords the ability to create cellular, organ and organismal systems which mimic these diseases, which provide an ideal setting in which to test various compounds for therapeutic activity. Particularly preferred compounds exhibit antidepressant effects by disrupting the interaction of Gsα-tubulin or induce a shift in the subcellular localization of Gsα from a tubulin-rich location to a region of less tubulin. In the screening assays of the present invention, the candidate compound may first be screened for basic biochemical activity—e.g., binding to a target molecule and then tested for its ability to induce antidepressant effects, at the cellular, tissue or whole animal level.

The present invention provides methods of screening for compounds that disrupt the Gsα-tubulin interaction. It is contemplated that this screening techniques will prove useful in the identification of compounds that induce anti-depressant effects.

In these embodiment, the present invention is directed to a method for determining the ability of a candidate compound to disrupt the Gsα-tubulin interaction, generally including the steps of:
a) providing a cell expressing tubulin and Gsα;
b) contacting said cell with a candidate modulator; and
c) monitoring said cell for change in a Gsα binding to tubulin or the cellular location of Gsα or another cellular property associated with antidepressant activity in the presence of said modulator.

To identify a candidate compound as being capable of disrupting the Gsα-tubulin interaction or exhibiting antidepressant activity in the assay above, one would measure or determine various characteristics of the cell, for example, an increase in adenylyl cyclase, activity, particularly outside of lipid rafts, would indicate a test compound that is capable of disrupting the Gsα-tubulin interaction. One would add the candidate compound to the cell and determine the response in the presence of the candidate compound. A candidate substance which modulates any of these characteristics is indicative of a candidate substance having modulatory activity. In the in vivo screening assays of the present invention, the compound is added to the cells, over period of time and in various dosages, and desired cellular response is measured.

### A. Candidate Compounds

As used herein the term “candidate compound” refers to any molecule that may potentially act as an inhibitor of the Gsα-tubulin complex. The candidate compound may be a protein or fragment thereof, a small molecule inhibitor, peptidomimetics or antibody. It may prove to be the case that the most useful pharmacological compounds will be compounds that are structurally related to other known antidepressant agents. Rational drug design includes not only comparisons with known antidepressant agents, but predictions relating to the structure of target molecules. Particularly useful compounds for use in rational drug design are those that will disrupt the interaction of Gsα with tubulin.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like tubulin, or a fragment thereof. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds molded of active, but otherwise undesirable compounds.

Candidate compounds may include fragments or, parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known antidepressants.

“Effective amounts” in certain circumstances are those amounts effective to disrupt the Gsα-tubulin interaction in a cell. Compounds that achieve significant appropriate changes in activity will be used.

B. In vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to tubulin or fragment thereof or microtubules is provided. In another embodiment, the screening of compounds that bind to Gsα or fragments thereof is provided.

The target may be either free in solution, fixed to support, such as a membrane, expressed in or on the surface of a cell. Either the target or the compound may be labeled, thereby permitting determining of binding. In another embodiment, the assay may measure the inhibition of binding of a target to a natural or artificial substrate or binding partner (such as tubulin and Gsα). Competitive binding assays can be performed in which one of the agents (Gsα, for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 94/03564. Large numbers of small peptide test compounds are synthesized on a solid substrate, such as plastic pins or some other surface. The peptide test compounds are reacted with, for example, tubulin and washed. Bound polypeptide is detected by various methods.

Purified target, such as tubulin or Gsα, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region to a solid phase.

C. In cyto Assays

Various cell lines can be utilized for screening of candidate substances. For example, rat glioma cells, such as C6-2B cells or human neuroblastoma cells (SK N SH), can be used to study various functional attributes of candidate compounds. In such assays, the compound would be formulated appropriately, given its biochemical nature, and contacted with a target cell. In addition, cells may be selected for assays of the invention for their endogenous Gs-coupled receptors or receptors. Further, the assays of the invention may be carried out with cells that are co-transfected with nucleic acids encoding GFP-Gsα (green fluorescent protein-Gsα).

Depending on the assay, culture may be required. As discussed above, the cell may then be examined by virtue of a number of different physiologic assays. Alternatively, molecular analysis may be performed in which the function of Gsα and related pathway may be explored. This involves assays such as those for protein expression, enzyme function, substrate utilization, mRNA expression (including differential display of whole cell or polyA RNA) and others. For cell-free assays, the Gsα-tubulin interaction can be assessed by using a solid-phase binding assay such as the SPOT assay described in Example 1.

D. In vivo Assays

The present invention particularly contemplates the use of various animal models. Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

Determining the effectiveness of a compound in vivo may involve a variety of different criteria. Such behaviors exhibited by the animal include, but are not limited to, reduced sleep disruption, reduced weight loss, improved reward insensitivity, reduced attention deficits, increased sexual activity, increased time of struggling in the forced swim test, increased sucrose preference and increased social interaction. Additional assays for measuring the effectiveness of antidepressant activity are provided in Willner, Trends Pharmacol Sci. 12(4):131-6, 1991 and Dekeyne, Therapie. 60(5):477-84, 2005. It also is possible to perform histologic studies on tissues from these animals, or to examine the molecular and morphological state of the cells.

### Compositions

In the sections above, the present invention describes various novel compositions for disruption of the Gsα-tubulin complex, also described are assays for identifying additional composition. It is contemplated that therapeutic compositions of the present invention will be useful in the intervention of various disease states such as for example, mood disorders such as depression and bipolar disorder, anxiety disorders such as phobia disorder, panic disorders, stress disorders and obsessive-compulsive disorders, and addiction to abusive drugs such as cocaine or opiates. Such agents may be used either alone or in combination with other therapeutic agents presently being used to control these disorders. In order to be used in such therapeutic indications, it will be preferable to prepare the compositions of the invention in pharmaceutically acceptable formats.

Also, it should be understood that it may well be that purified compositions that disrupts the Gsα-tubulin complex may be routinely prepared into pharmaceutically acceptable forms of the proteins once they are isolated from the media and/or cellular compositions described above. Generally, this will entail preparing compositions that are essentially free of pyrogens, as well as other impurities that could be harmful to humans or animals.

One will generally desire to employ appropriate salts and buffers to render the compositions stable and allow for uptake by target cells. Buffers also will be employed when recombinant cells or nucleic acids are introduced into a subject. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions produced by the present invention, its use in therapeutic compositions is contemplated. Supplementary active ingredients also can be incorporated into the compositions.

The compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The compositions produced using the present invention may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the compositions produced by the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. For example, parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose-response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, farther information will emerge regarding appropriate dosage levels and duration of treatment for specific diseases and conditions.

It will be appreciated that the pharmaceutical compositions and treatment methods employing such compositions may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey, ducks and geese.

In view of the foregoing discussion and by way of illustration of the invention, the examples describe methods for identifying peptides that disrupt the interaction of Gsα and tubulin, describes the characterization of the Gsα and tubulin binding sites and describes the heterotrimeric complex comprising Gsα and tubulin.

### EXAMPLES

### Example 1

### Material and Methods

### SPOT Membranes

Peptides were synthesized on to a cellulose membrane with PEG spacer (8×12 cm²) (AIMS Scientific Products, Braunschweig, Germany) via the C-terminal amino acid in sequential spots by the use of a SPOT synthesis kit (SIGMA genosys, St. Louis, Mo.) (Frank, J. Immunol. Methods 267: 13-26, 2002; Frank, Tetrahedron 48: 9217-9232, 1992). The peptides corresponded to the amino acid sequence of Gsα (Genbank accession number P04895, homo sapiens, Gsα long form; SEQ ID NO: 15), which was divided into overlapping peptides (12 amino acids in length with 7 amino acid overlap between sequential peptide, 73 total spots). Spot 1 corresponded to amino acids 1-12 and Spot 2 corresponded to residues 6-17 in the primary amino acid sequence (SEQ ID NO: 15), etc.

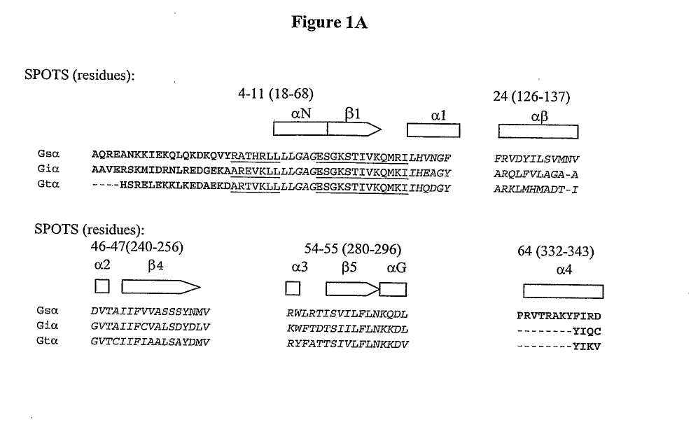

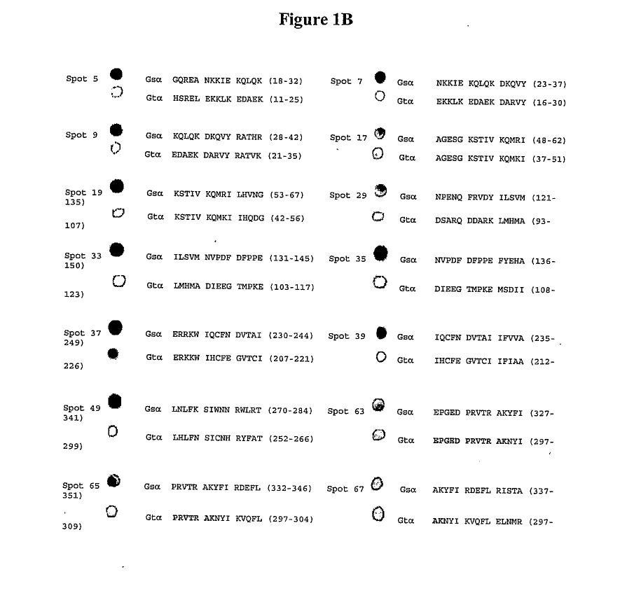

Another SPOT membrane was created based on the results of SPOT membrane 1, to compare Gsα-peptides that bound tubulin to Gtα-peptides, because Gtα has been shown to not bind tubulin. The sequence of the Gsα-peptides that were found to bind tubulin (with an approximate 5 amino acid extension added on both sides) was used as well as the corresponding amino acid sequence of Gtα. For instance, the Gsα sequence of SPOTS 4-11 in FIG. 1 was taken, the 5 amino acids from the Gsα sequence toward the N-terminal as well as C-terminal were added to the both ends of the sequence, and then the sequence was divided into overlapping 15 amino acid length peptides. These peptides were divided into overlapping peptides (15 amino acids in length with 10 amino acid overlap between sequential peptide, 70 total spots). Spot 1 corresponded to the 1^{st} 15 amino acids from Gsα that bound tubulin, spot 2 corresponded to sequence aligned amino acids from Gtα, spot 3 corresponded to a peptide shifted 5 amino acids toward the C-terminal end of that sequence from Gsα and spot 4 corresponded to the sequence aligned amino acids from Gtα, etc. Certain regions of Gsα lacked corresponding regions in Gtα. For these regions of Gtα, the corresponding amino acids of Gsα in these Gtα-peptides were substituted.

Membranes were blocked with TBS-containing 0.1% Tween-20 (TBS-T) with 2.5% milk for 1 hour, washed with TBS-T and incubated overnight at 4° C. with 150 nM tubulin in RIPA buffer (10 mM Tris-Cl, pH 7.4, 1% Titron-X-100, 1% Sodium Deoxycholate, 1% SDS and 500 mM NaCl). This assay was also carried out in the above buffer in the absence of SDS under nonreducing conditions. Next, the membranes were washed 3× with RIPA buffer and incubated with anti α-tubulin antibody (Sigma, St. Louis, Mo.), followed by the horseradish peroxidase conjugated secondary antibody (1 hour each at room temperature in the RIPA buffer containing 1% milk) and developed with enhanced chemiluminescence western blotting detection reagents (Amersham Biosciences).

For stripping the membranes, a previously described procedure was modified (14). Before reuse, the membrane was blocked and probed with the antibodies to verify that residual peptide-bound protein was stripped. Control experiments verified no specific binding of the primary antibody, secondary antibody or tubulin with the peptides after stripping the membranes. To further control for specific-binding, tubulin was incubated with equimolar Gsα (150 nM) at 37° C. for 1 hour to form protein complexes and next, the complexes were incubated with the SPOT membrane (SPOT membrane 1) as above. This prior incubation with Gsα prevented tubulin from binding to the immobilized peptides on SPOT membrane 1.

### Molecular Modeling

Because the crystal structure of Gsα was determined as a dimer (Sunahara et al., Science 278: 1943-1947, 1997), one of the Gsα molecules in the dimer was deleted along with all of its corresponding ligands (the PDB file is 1AZT). In the remaining Gsα molecule, the Mg²⁺ and PO₄⁴⁻ molecules were deleted and GTPγS was retained. For the structure of the tubulin dimer (Nogales et al., Cell 96: 79-88, 1999), the α-tubulin subunit was removed along with its corresponding nucleotide and taxol (the PDB file is 1TUB). For the remaining β subunit, GDP was retained and taxol was removed.

The docking algorithm, ZDOCK2.3 (Chen, et al., Proteins 52: 68-73, 2003) was used first for the unbound protein-protein docking where 2000 predictions were generated using β-tubulin as receptor and Gsα as ligand. ZDOCK was downloaded to a Linux system. Parameters were added to uniCHARM for GTPγS (GSP, as named in the original GsαPDB). The parameters used were from the uniCHARM file for GNP and the sulfur in GTPγS was used from the sulfur in CYS. ZDOCK uses a fast Fourier Transform algorithm. The protein-protein interface was evaluated by shape complementarity, desolvation energy, and electrostatics.

The 2000 complexes generated from ZDOCK were then submitted to ClusPro, http://nrc.bu.edu/cluster/ (Comeau et al., Bioinformatics 20: 45-50, 2004). ClusPro calculates pair-wise RMSD values to find neighbour complexes within 9 Å of another complex. These complexes were then clustered and the top 30 clusters were returned for further evaluation. To minimize the side chain clashes, the ranked complexes in the clusters were subjected to a minimization using CHARMM (Comeau et al., Bioinformatics 20: 45-50, 2004). These clusters were then ranked according to population in each cluster. The parameters for ClusPro were set in the advanced options section for filtering and clustering, with a radius of 9 Å, the electrostatic hits at 1500, and a return cluster output of 30. The representative complex for each particular cluster was the complex that is most centrally located in the array of complexes.

Further characterization of the top five complexes was performed by energy minimization of the ZDOCK/ClusPro-derived complexes. Each of the top 5 complexes was further examined for an additional 5000 cycles with the SANDER package within AMBER7 and a minimization energy score was determined. The buried surface area (BSA) of each complex was determined within the GRASP program, as to determine which complex has the largest contact area.

Visualization of protein structures was performed via GRASP (Nicholls et al., Proteins 11: 281-296, 1991) or SYBYL 6.9 (Tripos, Inc, St. Louis, Mo.) software.

### Protein Preparations

Ovine brain tubulin was prepared as previously described (Shelanski et al., Proc. Natl. Acad. Sci. USA. 70: 765-768, 1973). Briefly, the brains were obtained at a local slaughterhouse from freshly-killed animals and were placed on ice upon removal from animals. Purity of the prepared tubulin, as determined by SDS gel electrophoresis, was always greater than 95%. Nucleotide replacement on tubulin was performed as before (Popova et al., J. Neurosci. 20: 2774-2782, 2000), using charcoal to strip bound nucleotide. Protein concentrations were determined by Coomasie Blue binding (BioRad Protein Assay) with Bovine Serum Albumin as a standard (Bradford, Anal. Biochem. 72: 248-254, 1976).

### Example 2

### Gsα-Derived Peptides that Bind Tubulin

The SPOT technique was used to determine the specific domains on Gsα that bind to tubulin (Frank, J. Immunol. Methods 267: 13-26, 2002). To carry out this analysis, peptides corresponding to the primary Gsα sequence were covalently attached to a cellulose-based membrane. The membrane was probed with tubulin in the GDP or GTP bound states. Tubulin-GDP interacted with 9 peptides and tubulin-GTP interacted with 6 peptides. Probing was carried out with binding a tubulin specific antibody, therefore control experiments were performed to determine whether the primary or secondary antibody interact non-specifically with the peptides. No non-specific binding was detected on the SPOT membrane. After each experiment examining tubulin binding to the SPOT membrane, the membrane was stripped and probed with primary antibody followed by secondary antibody. Control experiments indicated no residual tubulin or antibody binding to the immobilized peptides.

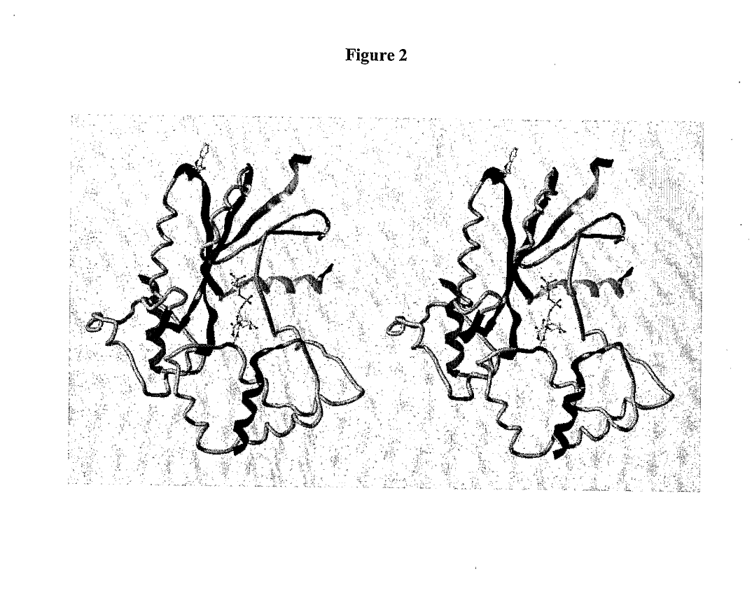

Tubulin binds with a high affinity (KD ≈130 nM) to Gsα and Giα, but does not bind to the photoreceptor G protein, Gtα(Wang et al., J. Biol. Chem. 265: 1239-1242, 1990). FIG. 1a shows the amino acids of the Gsα-peptide that exhibited binding to tubulin along with the corresponding amino acids from Giα and Gtα. In spots 4-11, the amino terminal region of Gsα, there are an additional 4 amino acids in Gsα and Giα1 that are not found in Gtα. Other interesting domains that interacted with tubulin, the α2-β4 and α3-β5 regions, are known to be important in the interaction with adenylyl cyclase (Tesmer et al., Science 278: 1907-1916, 1997). Furthermore, in the α3-β5 region, there is a tryptophan in Gsα (TRP281) and Giα1 that corresponds to a tyrosine in Gtα. This residue is located on the protein surface where solvent-exposed hydrophobic residues often contribute to protein-protein interactions (FIGS. 1 and 2).

The structure of Gα proteins includes two domains: a GTPase domain and an α-helical domain (Lambright et al., Nature 369: 621-628, 1994). Data from peptide binding (SPOT studies) suggest that the primary tubulin binding sites on Gsα are localized to the GTPase domain (FIG. 2). The GTPase domain of Gsα includes the switch regions (Lambright et al., Nature 369: 621-628, 1994): switch I (αF-β2); switch II (β3-α2-β4), and switch III (β4-α3), which are important for adenylyl cyclase activation and are structurally altered upon exchange of GDP for GTP (17). Two domains of Gsα that bound tubulin on the SPOT membrane, α2-β4 (spots 46-47, residues 240-256) and α3-β5 (spots 54-55, residues 280-296), are included in these regions.

To further test the binding of tubulin to the Gsα-peptides, the binding of these Gsα-peptides was compared to the binding of Gtα-Peptides, using a separate peptide-array membrane (see Example 1) because Gtα is known to not bind tubulin despite significant sequence and structural similarities to Gsα. Peptides from Gsα that had enhanced binding as compared to the corresponding Gtα peptide are shown (FIG. 1b). As before, this membrane was probed with tubulin in both the GDP and GTP bound state. Since we were probing for binding with antibodies, control experiments were also performed which demonstrated no non-specific binding of the primary or secondary antibodies or residual tubulin binding to the membrane following stripping (see Example 1). Distinct regions in the amino terminus and the switch II and III regions of Gsα bind tubulin but not the corresponding region of Gtα providing novel leads into which domains of Gtα may cause it to not bind tubulin (see FIG. 1b). These data also reconfirmed the binding shown with the first SPOT membrane even though the length of the peptides were longer in this membrane (15 vs. 12 amino acids), in which all the Gsα-peptides probed in this membrane bound to tubulin in one or the other (or both) nucleotide states of tubulin.

To carry out binding analysis by Surface Plasmon Resonance (SPR), quantitative analysis of peptide-tubulin interactions were performed on a BIAcore advance system (Pharmacia Biosensor AB, Uppsala, Sweden). Tubulin was immobilized in Hepes buffer pH 8.0 (50 mM Hepes, 0.1M NaCl, 1 mMEDTA, 1 mM DTT) at a flow rate of 10 μL/min on sensor chip CM4 according to the manufacturer's instructions. The immobilization level was approx. 300 RU (resonance units). Different concentrations of synthetic peptides (25-100 μM in Hepes buffer) were injected onto the flow cell at a flow rate of 30 μL/min. Sensograms were subtracted from buffer blank injections. The surface was regenerated by injection of 1M NaCl, 1% tritronX-100 in Hepes buffer (pH8.0). Sensograms were analzyed by the BIAevaluation 4.1 program (Pharmacia Biosensor AB).

### Example 3

### Analysis of the Gsα and Tubulin Complex

To further analyze the interaction between Gsα and tubulin, two protein-protein docking programs (ZDOCK (Chen, et al., Proteins 52: 68-73, 2003) and ClusPro (Comeau et al., Bioinformatics 20: 45-50, 2004) were used to generate and analyze protein complexes. These docking algorithms do not include conformational changes induced by the interactions; however, these programs have been successful in predicting near-native structural complexes for other systems (Chen, et al., Proteins 52: 68-73, 2003; Comeau et al., Bioinformatics 20: 45-50, 2004; Camacho et al., Proteins 52: 92-97, 2003). Using the structures of Gsα-GTPγS (Sunahara et al., Science 278: 1943-1947, 1997) and β-tubulin-GDP (Nogales et al., Cell 96: 79-88, 1999), 2000 potential complexes by ZDOCK were generated and were clustered with ClusPro. ClusPro created and ranked 30 clusters with the cluster ranking based on the number of similar complexes in each cluster. The complex that is most centrally located in each cluster was used to represent that cluster (Comeau et al., Bioinformatics 20: 45-50, 2004). In the docking programs, only the structure of the β-tubulin subunit was used, based on the assumption that Gα proteins interact only with this subunit because this subunit contains the exchangeable nucleotide that is likely involved in the transactivation mechanism (Wang et al., J. Biol. Chem. 265: 1239-1242, 1990; Rasenick et al., Methods Enzymol. 390: 389-403, 2004; Roychowdhury et al., J. Biol. Chem. 274:13485-13490, 1999).

After obtaining the final 30 complexes, the number of amino acids on Gsα that were in the interface of this protein-protein interaction were analyzed and were also predicted from the SPOT membrane (Table 2). Twenty-eight of the 30 complexes included some residues on Gsα that were in the protein interface that were also determined by this direct binding technique. Fourteen of the 30 complexes had over thirty percent of the residues in the interface predicted by the direct binding results of the SPOT membrane.

TABLE 2
Gsα and β-Tubulin Residues in the Protein-Protein Interface

| Complex (#) | Gsα | β-tubulin |
|---|---|---|
| 1 (17) | 3/15 | 10/21 |
| 2 (16) | 6/16 | 11/25 |
| 3 (15) | 0/10 | 14/24 |
| 4 (15) | 9/21 | 8/27 |
| 5 (14) | 3/17 | 12/20 |
| 6 (14) | 6/11 | 15/28 |
| 7 (13) | 6/12 | 13/22 |
| 8 (13) | 4/16 | 12/24 |
| 9 (12) | 3/19 | 0/27 |
| 10 (11) | 9/11 | 10/19 |
| 11 (11) | 3/26 | 0/27 |
| 12 (11) | 7/12 | 0/16 |
| 13 (11) | 8/20 | 16/29 |
| 14 (11) | 3/10 | 9/17 |
| 15 (11) | 0/20 | 4/19 |
| 16 (10) | 3/11 | 1/14 |
| 17 (10) | 3/10 | 10/17 |
| 18 (10) | 2/23 | 16/32 |
| 19 (9) | 2/13 | 10/15 |
| 20 (9) | 3/25 | 19/30 |
| 21 (9) | 3/16 | 12/25 |
| 22 (9) | 5/6 | 0/15 |
| 23 (9) | 4/21 | 10/17 |
| 24 (9) | 9/17 | 0/25 |
| 25 (8) | 5/7 | 7/15 |
| 26 (8) | 3/15 | 0/23 |
| 27 (8) | 7/14 | 0/13 |
| 28 (8) | 5/19 | 0/19 |
| 29 (8) | 3/19 | 0/23 |
| 30 (8) | 1/18 | 14/26 |

In Table 2, the number in parentheses in the left column indicates the number of ZDOCK-generated complexes that made up that particular cluster as determined by ClusPro. The numerator corresponds to number of residues in Gsα (middle column) or β-tubulin (right column) within 5 Å of the other protein that were detected from the SPOT membrane data (middle column) or predicted from a hypothetical β-tubulin interface (right column), respectively. The denominator corresponds to the total number of residues within the interface, as determined from that ZDOCK-generated complex. The residues that were less than 5 Å from another residue on the other protein were considered to be in the protein interface.

In FIG. 3A, the relative position of β-tubulin (for the top thirty complexes) to a fixed Gsα (represented as a whole molecule) is shown. Most of the potential β-tubulins cluster near the GTPase domain of Gsα. This indicates a favorable orientation of β-tubulin predicted by the docking programs to be located in the GTPase domain of Gsα. Further, the regions predicted by the SPOT membrane are highlighted on Gsα in FIG. 3A indicating many of the predicted interfaces of the 30 complexes are in this region.

The interactions that occur between tubulin dimers within microtubules (called interdimer interactions) have been well defined and occur at several residues surrounding the nucleotide on β-tubulin (27 amino acids have been defined) (Nogales et al., Cell 96: 79-88, 1999). It was hypothesized that Gsα binds close to the nucleotide binding site on α-tubulin and in a similar region to which the β-tubulin of another tubulin dimer would bind. Modeling of the top thirty complexes (FIG. 3B) with the relative position of Gsα, represented by circles, to a fixed β-tubulin, indicated that a majority of these docked Gsα molecules were located around the exchangeable nucleotide-binding site of β-tubulin. This is consistent with the above hypothesis. The residues in the interface of the top 30 complexes were analyzed to test this hypothesis. This analysis determined that most of the complexes did indeed contain these β-tubulin residues in the interface (FIG. 3B. and Table 3). For 18 of the 30 complexes, over 40% of the residues on β-tubulin in the interface were predicted by the above assumption. Of the top 10 complexes, only one (complex 9) did not contain any of these residues.

The top five complexes from the ZDOCK/ClusPro anaylsis were farther analyzed by energy minimization cycles (5000) and the buried surface area (BSA) of each of these complexes was then determined (Table 3). The energy values indicate that complex 2 has the lowest energy, followed by both complexes 1 and 3 being slightly higher in energy than 2, with 4 and 5 showing the highest energies of the five complexes. Complex 2 also has the most buried surface area at the interface, perhaps suggesting a better interaction of the two molecules.

TABLE 3
Minimization energies, Buried Surface Area (BSA), and interacting regions
between the different domains of β-tubulin and Gsα for Complexes 1-5.

| Complex | Energy | BSA(Å²) | β-Tubulin | Gsα |
|---|---|---|---|---|
| 1 | −11,515 | 3326 | H1(11) | α3-β5(280-283) |
| | | | B2-H2(69-74) | α3-β5(280-281) |
| | | | B3-H3(98-105) | α3-β5(278-283), α4-β6(351-356) |
| | | | B4-H4(142) | α4-β6(356) |
| | | | H5(179-185) | α4-β6(354-356) |
| | | | H11-H12(404-411) | α4-β6(348-355), α3(277) |
| 2 | −12,207 | 3954 | H1(11) | α3-β5(283), α4-β6(356) |
| | | | B2-H2(71-77) | α3-β5(283-284), α4-β6(354-358) |
| | | | B3-H3(93-113) | α5(386-391), α3-β5(280-285, N- |
| | | | | term(38) |
| | | | B4-H4(142-143) | α3-β5(280) |
| | | | B5-H5(178-185) | α3-β5(280-281) |
| | | | H11-H12(407-411) | α2(235-239), α3-β5(281) |
| 3 | −11,475 | 3552 | H1(11-15) | C-term(391) |
| | | | B3-H3(95-110) | α3(283), β6(354-358), C- |
| | | | | term(389) |
| | | | B4-H4(142-143) | C-term(389) |
| | | | B5-H5(176-185) | β6(358-360), C-term(385-389) |
| | | | H7(224) | C-term(388) |
| | | | H11-H12(404-411) | α4-β6(352-358) |
| 4 | −11,079 | 3397 | H1(11-25) | α2(235-240), N-term(35-38) |
| | | | B2-H2(71-83) | α2(236-239), β5(207-211), |
| | | | | α2(220-236), N-term(35-42) |
| | | | H7(220-232) | C-term(389-391), α3-β5(280-284), |
| | | | | α4-β6(355-356), α2(239-240), |
| | | | | N-term(38) |
| | | | B7-H9(278) | C-term(391) |
| 5 | −10,936 | 3132 | H1(11-15) | αG-α4(309-317), α4(336) |
| | | | B2-H2(71-76) | αG-α4(307-310) |
| | | | B3-H3(96-105) | αG-α4(304-307), |
| | | | | α4(331-332) |
| | | | B5-H5(175-180) | αG-α4(320-325) |
| | | | H7(224) | αG-α4(318-320) |
| | | | H11-H12(407) | αG-α4(329-331) |

In Table 3, domain interactions between the two proteins were determined by identifying residues that were less than 4 Å from another residue on the other protein and were considered to be potential protein contacts. Based on these residue-residue contacts, the domains that defined these residues are shown. In parentheses, the residue number or the range of residues that contributed to the residue-residue contacts are indicated. For β-tubulin, cc-helices are listed as H1-H12 and β-sheets are listed as B1-B10 (20).

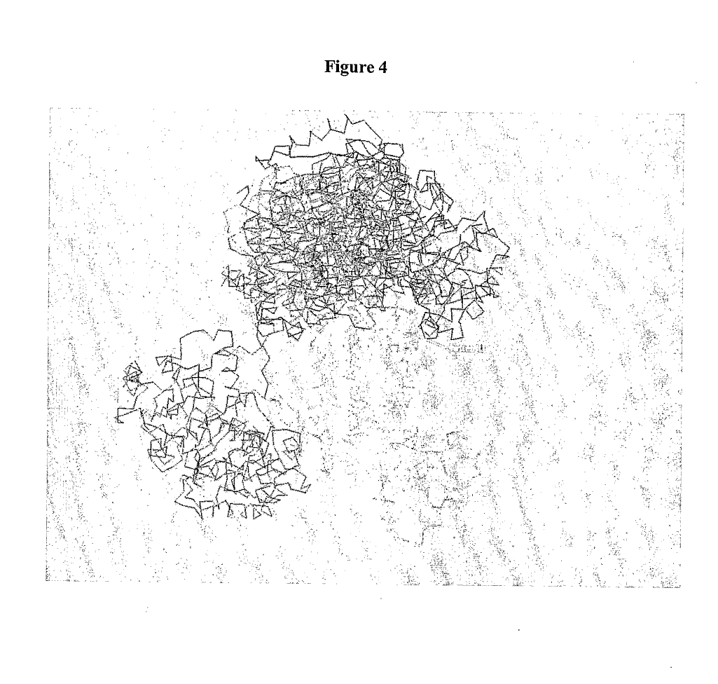

FIG. 4 shows an α-carbon alignment of the top five complexes, with a fixed Gsα and the relative position of corresponding β-tubulin for the top 5 complexes. As apparent, the top four complexes are similar in the location that the β-tubulin interacts with the Gsα. Complex 5, is in a different location on the Gsα, and is significantly higher in energy than the other four complexes, and therefore, presumably, an unfavorable complex. Complex 4 is significantly higher in energy than complexes 1-3, but has a number of similar regions of interactions with the top three complexes (Table 2). The top complexes have a number of common contact regions between Gsα and β-tubulin, in particular, in the regions of α3-β5 and α4-β6 loops of Gsα (Table 3)

Prior to this study, chimeric proteins comprised of Gsα, Giα1 and Gtα were used to define the domains on Gα proteins that are important in the interaction with tubulin. One study (Popova et al., J. Biol. Chem. 269: 21748-21754, 1994) demonstrated that the amino terminus (residues 1-63) of Gsα may be important for tubulin to activate adenylyl cyclase. The data provided herein indicated that the amino terminus plays a role in the binding to tubulin. For the top five complexes, only complex 4 (Table 2) includes a significant portion of the amino terminus of Gsα in the interface, complex 2 includes a small portion of the amino terminus of Gsα in the interface. The simplest reason for seeing less amino terminal involvement than expected in the top five complexes is that the structure of Gsα used in the molecular docking studies was missing portions of the amino terminus. This absent region of the amino terminus included the following residues predicted by the SPOT technique to be important: ALA18-LYS34 and ASN66-PHE68 of SEQ ID NO: 15. Therefore, a significant portion of the amino terminus of the Gsα was not available for analysis by the protein-protein docking programs.

In another study (Chen et al., J. Biol. Chem. 278: 15285-15290, 2003), it was demonstrated that one of the major Giα1-tubulin interacting domains was between residues 237-270 of Giα1 (which corresponds to residues 253-293 in Gsα (SEQ ID NO: 15), regions β4-α3-β5). However, this was not the only region involved in binding of the two molecules (Chen et al., J. Biol. Chem. 278: 15285-15290, 2003). It is assumed that Gsα and Giα1 complex with tubulin at a similar interface, regions α3-β5 of the Gα protein family appear important for interaction with tubulin. As seen in Table 2, the α3-β5 region of Gsα is included in the interface of 4 of the 5 top . complexes indicating that the predicted models agree with the data from both SPOT membrane and Gtα-Giα1 chimeras (Chen et al., J. Biol. Chem. 278: 15285-15290, 2003). Further, the switch II region (α2-β4) is in the interface of complex 4 (and complex 2), which is another domain predicted from the binding data and studies with chimeric Gα proteins (Chen et al., J. Biol. Chem. 278: 15285-15290, 2003).

Previous studies on G protein-tubulin interaction showed that the certain G protein heterotrimers could form a complex with tubulin (Wang et al., Biochemistry 30: 10957-10965, 1991). However, the deduced structure of Giα1β1γ2 (Wall et al., Cell 83: 1047-1058, 1995) suggests that most of the switch II region (including the following domains, α2-β4) as well as the amino terminus of Giγ1 are involved in the interface with the β1γ2 subunits. This interface on the Gα subunit has many similarities to our proposed interface on Gsα to tubulin, which could be interpreted to mean that Gα and tubulin could not bind contemporaneously to Gβγ. A report (Ford et al., Science 280: 1271-1274, 1998) showing interactions of these domains on Gα with effector and Gβγ has data consistent with the possibility that both Gβγ or effector (and, by inference, tubulin) can bind to Gα at the same time (although this was not the conclusion of the authors). The data described herein showed some overlap between sites of Gα for Gβγ and effector. In fact, while there was overlap, this observation, coupled with the movable nature of the switch II and switch III regions, gives rise to the possibility that Gα might enjoy contemporaneous-association with Gβγ and effector (or tubulin). It is also noteworthy that some recent reports suggest that physical dissociation of Gα and Gβγ might not be a prerequisite of Gα activation (Bunemann et al., Proc. Natl. Acad. Sci. U S A. 100: 16077-16082, 2003; Gales et al., Nat Methods 2: 177-184, 2005).

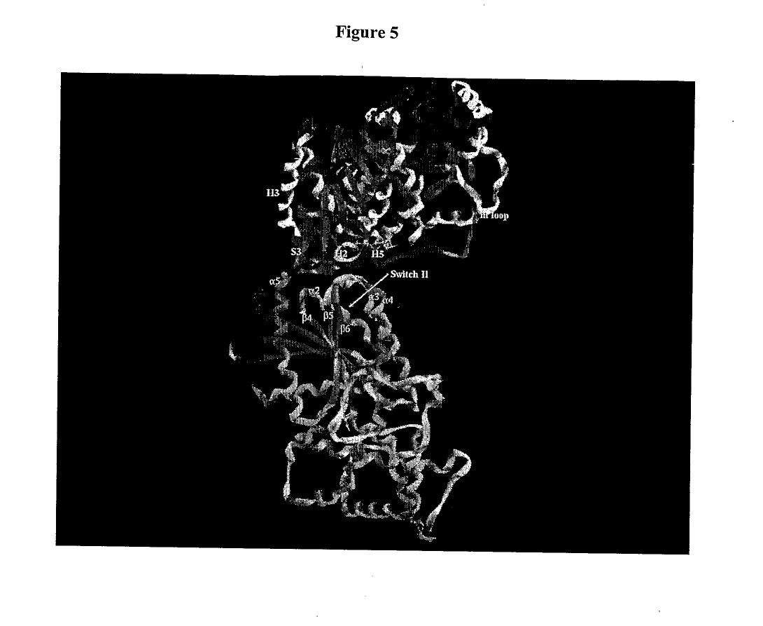

Comparison of the interacting surfaces of Gsα and β-tubulin for the top five complexes in Table 2 indicated similar interactions. However, in FIG. 5, complex 2 is shown, because this model has the lowest energy as seen in Table 2. In this model, the switch II region (α2-β4) and the amino terminal of Gsα both contain contacts to the H1 and H2 regions of β-tubulin (FIG. 5). Interestingly, the amino terminal of Gsα (seen directly under the switch II region, FIG. 5) extends directly into the nucleotide-binding pocket of β-tubulin.

What is claimed:

1. A method of disrupting the complex of Gsα and tubulin in a cell comprising inhibiting the interaction of Gsα and tubulin within said cell by
contacting tubulin with a molecule that inhibits the interaction of Gsα and tubulin, wherein the molecule binds to tubulin within at least one region selected from the group consisting of the nucleotide binding site, H1 region or H2 region of tubulin; or
contacting Gsα with a molecule that inhibits the interaction of Gα and tubulin, wherein the molecule binds to Gsα within at least one region selected from the group consisting of the α3-β5 region, α4-β6 region, α2-β4 region or the amino terminus of Gsα.

2. The method of claim 1, wherein the molecule is selected from the group consisting of a peptide, small molecule, antibody or peptidomimetic.

3. The method of claim 2 wherein the peptide comprises one of the amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14.

4. The method of claim 2, wherein the antibody is a single chained antibody.

5. The method of claim 2, wherein the antibody is a monoclonal antibody.

6. A composition comprising a molecule that inhibits the interaction of Gsα and tubulin.

7. The composition of claim 6, wherein the molecule binds tubulin within at least one region selected from the group consisting of the nucleotide binding site, H1 region or H2 region of tubulin.

8. The composition of claim 6, wherein the molecule binds Gsα within at least one region selected from the group consisting of α3-β5 region, α4-β6 region, α2-β4 region or the amino terminus of Gsα.

9. The composition of any one of claims 6-8, wherein the molecule is selected from the group consisting of a peptide, small molecule, antibody or peptidomimetic.

10. The composition of claim 9, wherein the peptide comprises one of the amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14.

11. The composition of claim 9, wherein the antibody is a single chained antibody.

12. The composition of claim 9, wherein the antibody is a monoclonal antibody.

13. A method of treating a mood or anxiety disorder comprising administering a composition of any one of claims 6-12.

14. A method of identifying modulators of the interaction of Gsα and tubulin comprising
contacting a cell expressing Gsα and tubulin with a candidate compound, and
monitoring said cell for modulation of Gsα binding to tubulin,
wherein a candidate compound that reduces the binding of Gsα to tubulin is an inhibitor of the interaction of Gα and tubulin, and a candidate compound that increase the binding of Gsα to tubulin is a agonist of the interaction of Gsα and tubulin.
